(19) United States
(12) Patent Application Publication
(10) Pub. No.: US 2003/0114787 A1
(43) Pub. Date: Jun. 19, 2003

(54) Wearable peritoneal dialysis system

(76) Inventor: Victor Gura, Beverly Hills, CA (US)

Correspondence Address: David E. Heisey, Esq., LUCE, FORWARD, HAMILTON & SCRIPPS LLP, Suite 2600, 600 West Broadway, San Diego, CA, 92101, US

(21) Appl. No.: 10/015,542

(22) Filed: Dec. 13, 2001

(51) Int. Cl. A61M 1/00

(52) U.S. Cl. 604/29; 604/28

(57) ABSTRACT

A peritoneal dialysis system including a peritoneal dialyzer that utilizes fresh dialysate to remove impurities from the blood of the patient and a plurality of contoured sorbent devices connected in series for regenerating the spent dialysate; wherein the sorbent devices are adapted to be worn on a portion of the body of a patient

### BACKGROUND OF THE INVENTION

The present invention is directed to peritoneal dialysis systems, and more particularly to a peritoneal dialysis system that may be continuously worn by a patient.

### BACKGROUND OF THE INVENTION

Dialysis treatment is usually performed using hemodialysis or peritoneal dialysis. Hemodialysis is a process by which toxins are removed from the blood using a filtering membrane such as a dialyzer. Peritoneal dialysis is another form of dialysis treatment, wherein the patient's peritoneal membrane (abdominal lining) is used as the filter such that sterile dialysate is introduced into the peritoneal cavity (abdomen) through a permanent tube. Fresh dialysate circulates through the peritoneal cavity to draw impurities from surrounding blood vessels in the peritoneum, and spent dialysate is then drained from the peritoneal cavity. Advantageously, peritoneal dialysis eliminates the need for blood anticoagulants and access to blood vessels since the patient's blood is never externalized.

Typically, dialysis is administered in intermittent three to four hours sessions, which take place two or three times per week. However, there exists a growing body of research that prefers continuous dialysis over intermittent dialysis since far more toxins can be removed from the blood using continuous dialysis seven days a week, twenty-four hours a day. Some advantages of continuous dialysis include a decreased rate of morbidity and expected mortality, a decrease in the amount of medications required and a decrease in fluid intake and dietary restrictions.

During dialysis, recirculation of the dialysate usually requires about 120 liters of fresh dialysate for a 4-hour session. But, a regenerating sorbent device (such as the REDY sorbent cartridge) can be used to purify spent dialysate so that only 6 liters of fresh dialysate are necessary for a 4-hour dialysis session. However, most contemporary sorbent devices, including the REDY sorbent cartridge, are conically shaped, bulky and generally unsuitable to be worn on the body of a dialysis patient. Therefore, there is a substantial need for a wearable peritoneal dialysis system, which can which can be used continually, 24 hours a day, seven days a week.

### SUMMARY OF THE INVENTION

The present invention solves the problems associated with conventional peritoneal dialysis systems by providing a continuously wearable peritoneal dialysis system having a plurality of non-bulky, flexible sorbent devices, which may be comfortably worn on the body of a patient.

One aspect of the present invention involves a peritoneal dialysis system including a peritoneal dialyzer that utilizes dialysate to remove impurities from the blood of the patient and a plurality of sorbent devices for regenerating the dialysate, wherein the sorbent devices are adapted to be worn on the body of a patient.

Another aspect of the present invention involves a peritoneal dialysis system including a plurality of sorbent devices for regenerating the dialysate, wherein the sorbent devices are connected in series.

A further aspect of the present invention involves a peritoneal dialysis system including a plurality of sorbent devices for regenerating the dialysate, wherein the sorbent devices are connected in parallel.

An additional aspect of the present invention involves a peritoneal dialysis system including a plurality of sorbent devices for regenerating the dialysate, wherein each of the sorbent devices has a flexible casing adapted to conform to the body contour of the patient.

Yet another aspect of the present invention involves a peritoneal dialysis system including a plurality of sorbent devices for regenerating the dialysate, wherein the number of sorbent devices may be varied to reflect different dialysis prescriptions.

A further aspect of the present invention involves a peritoneal dialysis system including a plurality of sorbent devices for regenerating the dialysate, further including a side port for the infusion of additives into the dialysate from a plurality of additive reservoirs, wherein the additives may include sodium citrate, calcium, potassium or sodium bicarbonate.

An additional aspect of the present invention involves a peritoneal dialysis system including a plurality of sorbent devices for regenerating the dialysate, wherein the sorbent devices may include activated charcoal, urease, zirconium phosphate, hydrous zirconium oxide or activated carbon.

Another aspect of the present invention involves a method of continuous removal of impurities from the blood of a patient using a wearable peritoneal dialysis system including infusing fresh dialysate into the peritoneal cavity of the patient so that the impurities in the blood are diffused across the peritoneal membrane and into the dialysate, and removing the spent dialysate from the peritoneal cavity and regenerating the dialysate using a plurality of sorbent devices that are worn on the body of a patient.

Further applicability of the present invention will become apparent from a review of the detailed description and accompanying drawings. It should be understood that the description and examples, while indicating preferred embodiments of the present invention, are not intended to limit the scope of the invention, and various changes and modifications within the spirit and scope of the invention will become apparent to those skilled in the art.

### BRIEF DESCRIPTION OF THE DRAWINGS

The present invention will become more fully understood from the detailed description given below, together with the accompanying drawings, which are given by way of illustration only, and are not to be construed as limiting the scope of the present invention. In the drawings:

FIG. 1 is a perspective view of the peritoneal dialysis system worn around the waist of a patient according to the present invention.

FIG. 2 is a front view of the peritoneal dialysis system of FIG. 1 after being detached from the patient.

FIG. 3 is a perspective view of the additive pump section of the peritoneal dialysis system according to the present invention.

FIG. 4 is a perspective view of a first embodiment of the sorbent section of the peritoneal dialysis system according to the present invention.

FIG. 5 is a perspective view of a second embodiment of the sorbent section of the peritoneal dialysis system according to the present invention.

FIG. 6 is a perspective view of a variation of the second embodiment of the sorbent section of the peritoneal dialysis system according to the present invention.

FIG. 7 is a top view of a casing of a sorbent device of the peritoneal dialysis system according to the present invention.

### DETAILED DESCRIPTION OF THE PREFERRED EMBODIMENTS

Before starting a description of the Figures, instructions for interpreting the words and phrases of this patent document will be provided. More particularly, many jurisdictions allow a patentee to act as its own lexicographer, and thereby allow the patentee to provide instructions in a patent document as to how the words, terms and phrases of the document are to be interpreted as a legal matter. For example, in the United States, the prerogative of the patentee to act as its own lexicographer has been solidly established based on statutory and case law. Accordingly, the following section provides rules for interpreting the words, terms and phrases of this particular patent document.

Interpretive Rules

Rule 1: There is a “Specially Defined Terms” section set forth below. Only words, terms or phrases that are explicitly defined in the Specially Defined Terms are to be considered to have a special definition, and, of course, the explicit definition provided herein is to serve as the definition for these terms. Accordingly, sources such as the patent specification and extrinsic evidence shall not be used to help define these terms—the explicitly provided definitions shall control.

Rule 2: If a word, term or phrase is not specially defined, then its definition shall be determined in the first instance by resort to dictionaries and technical lexicons that either exist as of the time this patent document is filed. (See definition of “dictionaries and technical lexicons” below in the Specially defined Terms section.) It is acknowledged that dictionaries and technical lexicons often provide alternative definitions. Also, definitions provided in different dictionaries and different lexicons often differ and are not always entirely consistent. In that case, it must be decided which definition is in best accordance with this document. Rules 3 and 4, set forth below, provide some guidelines for choosing between alternative definitions for a word, term or phrase.

Rule 3: The role of the specification (other than the Specially Defined Terms section) as an interpretive or definitional aid shall be limited to helping choose between alternative definitions that meet the requirements of Rule 2 (above).

Rule 4: The role of extrinsic evidence (e.g., expert witnesses) as an interpretive or definitional aid shall be limited to helping choose between alternative definitions that meet the requirements of Rule 2 (above).

Specially Defined Terms

the present invention: means at least some embodiments of the present invention; references to various feature(s) of the “present invention” throughout this document do not mean that all claimed embodiments or methods include the referenced feature(s).

dictionaries and/or technical lexicons: any document whose primary purpose is the definition of words, terms and/or phrases; on the other hand, documents that merely discuss, explain or provide examples of devices or methods, without purporting to provide definitions of specific words, phrases or terms, are not to be considered as dictionaries and/or technical lexicons.

hemodialysis: a process by which microscopic toxins are moved from one side of a filtering membrane (such as a dialyzer, e.g.) to another, wherein waste products and excess chemicals (including, but not limited to electrolytes) in the blood pass through the membrane into a solution (such as dialysate, e.g.) that does not contain those toxins.

peritoneal dialysis: an alternative treatment option to hemodialysis, wherein the peritoneal membrane is used as a filter such that sterile dialysate is introduced into the peritoneal cavity through a permanent tube placed in the peritoneal cavity. Fresh dialysate circulates through the peritoneal cavity to draw impurities from surrounding blood vessels in the peritoneum, and spent dialysate is then drained from the peritoneal cavity.

dialysate: a fluid used for dialysis that may consist of a mixture of water, glucose, and certain elements (including, but not limited to electrolytes). During dialysis, waste products and excess chemicals in the blood pass through a filtering membrane (such as a dialyzer, e.g.) into the dialysate fluid.

dialyzer: a filtering membrane used to filer waste products and excess chemicals (including, but not limited to electrolytes) during dialysis. Typically, a dialyzer is an artificial kidney that contains many hollow membrane fibers surrounded by dialysate. While blood flows inside of the hollow membranes, toxins from the blood move through the membrane wall and into the dialysate. The purified blood remains inside the hollow membranes and is returned to the body.

To the extent that the definitions provided above are consistent with ordinary, plain and accustomed meanings (as generally evidenced, inter alia, by dictionaries and/or technical lexicons), the above definitions shall be considered supplemental in nature. To the extent that the definitions provided above are inconsistent with ordinary, plain and accustomed meanings (as generally evidenced, inter alia, by dictionaries and/or technical lexicons), the above definitions shall control. If the definitions provided above are broader than the ordinary, plain and accustomed meanings in some aspect, then the above definitions will control at least in relation to their broader aspects.

To the extent that a patentee may act as its own lexicographer under applicable law, it is hereby further directed that all words appearing in the claims section, except for the abovedefined words, shall take on their ordinary, plain and accustomed meanings (as generally evidenced, inter alia, by dictionaries and/or technical lexicons), and shall not be considered to be specially defined in this specification. Notwithstanding this limitation on the inference of “special definitions,” the specification may be used to evidence the appropriate ordinary, plain and accustomed meanings (as generally evidenced, inter alia, by dictionaries and/or technical lexicons), in the situation where a word or term used in the claims has more than one alternative ordinary, plain and accustomed meaning and the specification is helpful in choosing between the alternatives.

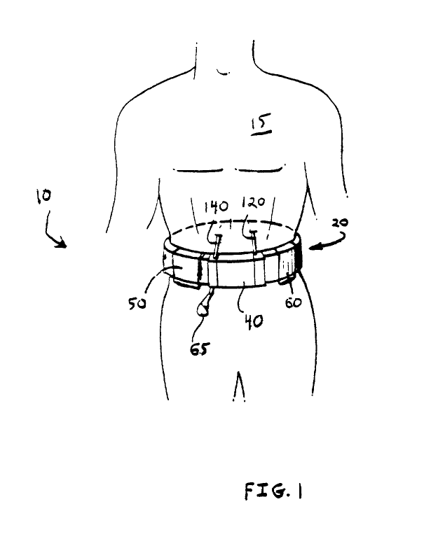

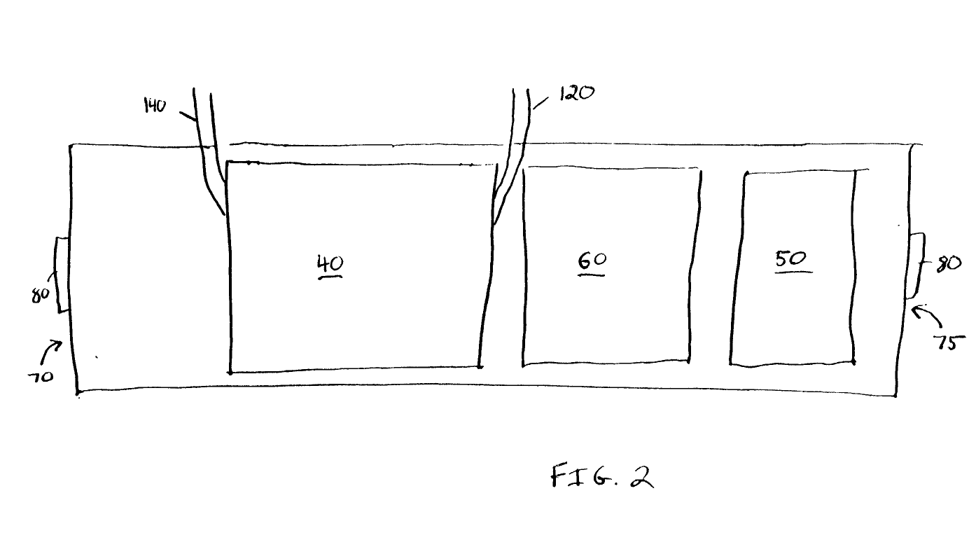

Referring to FIGS. 1 and 2, a peritoneal dialysis system 10 includes a belt 20 dimensioned to be worn about a portion of the body of a dialysis patient 15, and a peritoneal cavity or dialyzer within the patient's abdomen. The belt 20 is divided into a number of sections including a sorbent section 40, an additive pump section 50 and an electronic control section 60 that includes a microprocessor and batteries to power device 10. Fresh dialysate 100 is introduced into the peritoneal cavity through a regenerated dialysate inlet tube 120 and spent dialysate 100 is removed through a spent dialysate outlet tube 140.

Referring to FIG. 2, the belt 20 includes a pair of end portions 70, 75, which are secured together by a conventional belt fastener 80 such as a buckle, snaps, buttons or hook and loop fasteners. Although the belt 20 is worn about the waist of the patient 15, it should be understood to those of ordinary skill in the art that the belt 20 may, alternatively, be worn about other portions of the patient's body, such as over a shoulder of the patient, for example.

As would be known to one of ordinary skill in the art, the peritoneal cavity or dialyzer includes a semiporous peritoneal membrane, which, during peritoneal dialysis, separates the dialysate 100 from the patient's blood 130. Impurities in the blood 130 are diffused across the semiporous peritoneal membrane into the dialysate 100. Spent dialysate 100 then flows out of the peritoneal cavity, through a spent dialysate outlet tube 140 and into the sorbent section 40. Alternatively, a double lumen catheter can be used to provide inflow and outflow, thereby taking the place of the spent and regenerated dialysate tubes 120,140. Upon flowing into the sorbent section 40, the spent dialysate 100 is regenerated and reinfused into the peritoneal cavity of the patient 15.

Excess fluid is removed from the spent dialysate 100 through a volumetric chamber 155 and into a waste receiver 65, which is to be periodically emptied by the patient via tap 175. A microprocessor in the electronic section 60 determines the rate and amount of fluid removal through volumetric chamber 155.

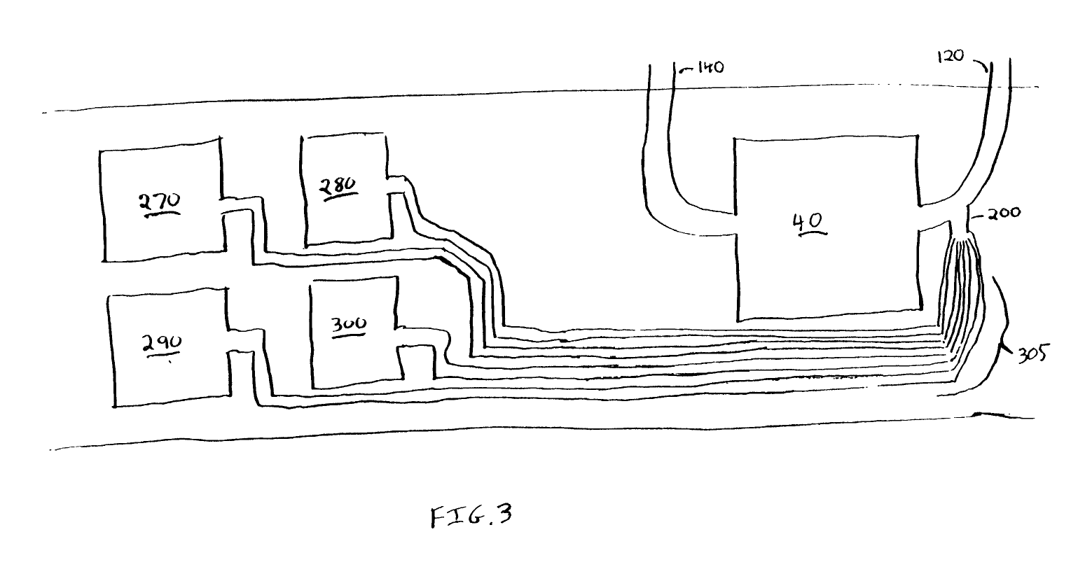

As best seen in FIG. 3, the regenerated dialysate inlet tube 120 includes a side port 200 for the infusion of additives, which are forced into the dialysate 100 from a plurality of additive pumps 270, 280, 290, 300. Piston, suction or roller pumps can be employed for this purpose. Each additive pump 270, 280, 290, 300 forces a controlled amount of respective additive into the dialysate, wherein the rate of infusion of each additive is controlled electronically by the microprocessor in the electronic control section 60. In a known manner, a physician can use the electronic control section 60 to set the rate of infusion for each additive to correspond to a predetermined dose for each additive. Since the additives cannot be mixed together prior to infusion in the blood 130, they have separate circuits 305. Typical additives include, but are not limited to, sodium citrate, calcium, potassium and sodium bicarbonate.

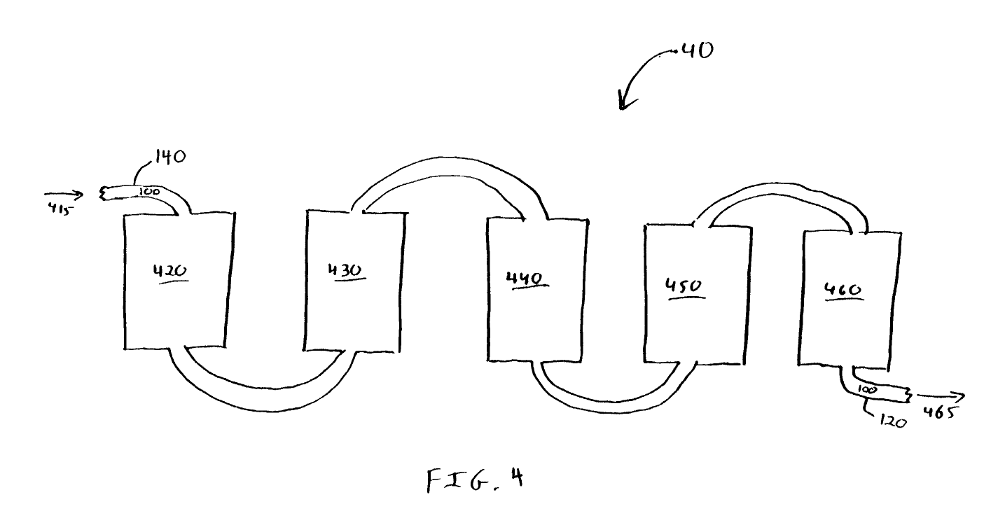

Referring to FIG. 4-6, in the sorbent section 40, as indicated by arrow 415, spent dialysate 100 flows from the peritoneal cavity 110 through spent dialysate outlet tube 140 and into a plurality of sorbent devices 420, 430, 440, 450, 460. As indicated by arrow 465, the regenerated dialysate 100 then flows through tube 120 and back into the peritoneal cavity 110. Preferably, the sorbent devices 420, 430, 440, 450, 460 comprise a series of sorbent cartridges 420, 430, 440, 450, 460 for regenerating the spent dialysate 100. By regenerating the dialysate 100 with sorbent cartridges 420, 430, 440, 450, 460, the peritoneal dialysis system 10 of the present invention requires only a small fraction of the amount of dialysate 100 of a single-pass dialysis system. Importantly, each sorbent cartridge 420, 430, 440, 450, 460 is a miniaturized sorbent cartridge 420, 430, 440, 450, 460 containing a distinct sorbent.

Referring to FIG. 4, in a first embodiment of the sorbent section 40, there are five sorbent cartridges 420, 430, 440, 450, 460 including an activated charcoal cartridge 420, a urease cartridge 430, a zirconium phosphate cartridge 440, a hydrous zirconium oxide cartridge 450 and an activated carbon cartridge 460. Those of ordinary skill in the art will recognize that these sorbents are similar to the sorbents employed by the commercially available Recirculating Dialysis (REDY) System. However, in the REDY System, the sorbents are layers of a single cartridge. By contrast, the sorbents of the present invention are each part of a distinct sorbent cartridge 420, 430, 440, 450, 460 such that each cartridge 420, 430, 440, 450, 460 may, conveniently, be replaced and disposed of independently of the other cartridges 420, 430, 440, 450, 460. As one of ordinary skill in the art would understand, activated charcoal, urease, zirconium phosphate, hydrous zirconium oxide and activated carbon are not the only chemicals that could be used as sorbents in the present peritoneal dialysis system 10. In fact, any number of additional or alternative sorbents could be employed without departing from the scope of the present invention.

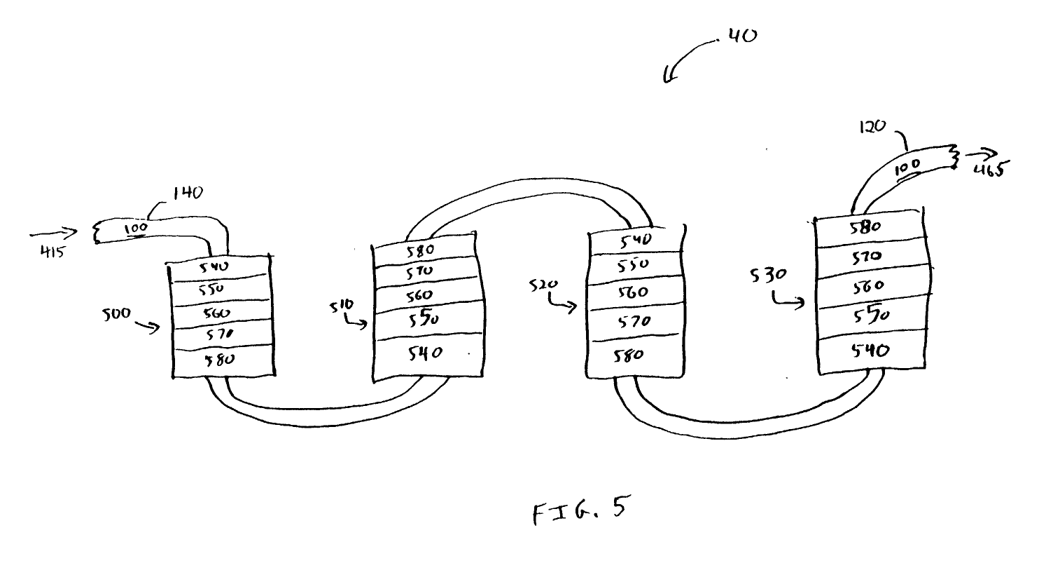

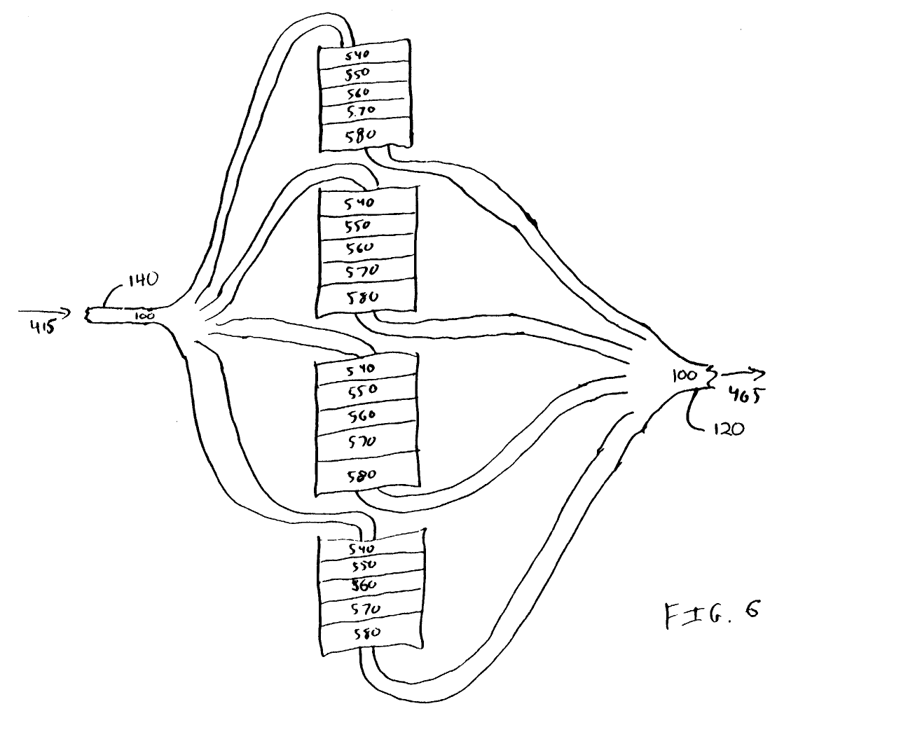

Referring to FIGS. 5 and 6, in a second embodiment of the sorbent section 40, there are a plurality of sorbent cartridges 500, 510, 520, 530, wherein each cartridge 500, 510, 520, 530 includes a plurality of sorbent layers 540, 550, 560, 570, 580: an activated charcoal layer 540, a urease layer 550, a zirconium phosphate layer 560, a hydrous zirconium oxide layer 570 and an activated carbon layer 580. The cartridges 500, 510, 520, 530 may be in series as depicted in FIG. 5 or may be in parallel as depicted in FIG. 6. In this embodiment, the number of sorbent devices may be varied to correspond with different dialysis prescriptions.

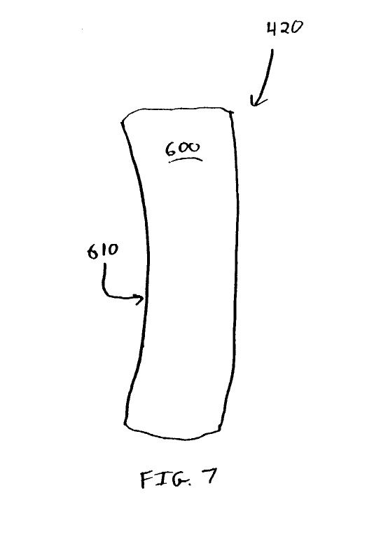

Referring to FIG. 7, each of the previously described sorbent cartridges 420,430,440,450,460,500,510,520,530 is a miniature cartridge having a flexible casing 600 adapted to conform to the body contour of the patient. In addition, the body-side wall 610 of each casing 600 is concave to further correspond to bodily curves. The casing 600 can be made of any suitable material having adequate flexibility for conformance to the portion of the body to which it is applied. Suitable materials include, but are not limited to polyurethane and poly vinyl chloride.

A method of continuous removal of impurities from the blood of a patient using a wearable peritoneal dialysis system will now be described. The method includes introducing fresh dialysate into the peritoneal cavity 110 of the dialysis patient through inlet tube 120 so that uremic toxins and other impurities in the blood are diffused across the semiporous peritoneal membrane 125 and into the dialysate 100, removing the spent dialysate 100 from the peritoneal cavity 110 through outlet tube 140 into the sorbent area 40, and regenerating the dialysate 100 using a plurality of sorbent devices 420, 430, 440, 450, 460 worn on the body of patient 15. The method may also include the step of varying the number of sorbent devices 420, 430, 440, 450, 460 to reflect different dialysis prescriptions. Preferably, these sorbent devices 420, 430, 440, 450, 460 are replaceable cartridges 420, 430, 440, 450, 460 with flexible casings 600 designed to conform to the shape of the body part to which they are applied.

Many variations on the above-described invention are possible. Such variations are not to be regarded as a departure from the spirit and scope of the invention, but rather as subject matter intended to be encompassed within the scope of the following claims, to the fullest extent allowed by applicable law.

What is claimed is:

1. A peritoneal dialysis system for a patient, comprising:
a peritoneal dialyzer that utilizes dialysate to remove impurities from the blood of the patient; and
a plurality of sorbent devices for regenerating the dialysate, wherein the sorbent devices are adapted to be worn on the body of a patient.

2. The peritoneal dialysis system of claim 1, wherein the sorbent devices are connected in series.

3. The peritoneal dialysis system of claim 1, wherein each of the sorbent devices has a flexible casing adapted to conform to the body contour of the patient.

4. The peritoneal dialysis system of claim 1, wherein the number of sorbent devices may be varied to reflect different dialysis prescriptions.

5. The peritoneal dialysis system of claim 1, further including a regenerated dialysate inlet tube leading into the peritoneal dialyzer and a spent dialysate outlet tube leading out of the peritoneal dialyzer.

6. The peritoneal dialysis system of claim 5, wherein the regenerated dialysate inlet tube includes a side port for the infusion of additives.

7. The peritoneal dialysis system of claim 6, wherein the additives are pumped into the dialysate from a plurality of additive reservoirs.

8. The peritoneal dialysis system of claim 6, wherein the rate of infusion of each additive is controlled electronically.

9. The peritoneal dialysis system of claim 6, wherein the additives are chosen from the group consisting of: sodium citrate, calcium, potassium and sodium bicarbonate.

10. The peritoneal dialysis system of claim 5, wherein the spent dialysate tube leads into the plurality of sorbent devices and the regenerated dialysate tube leads out of the plurality of sorbent devices.

11. The peritoneal dialysis system of claim 1, wherein the sorbent devices comprise replaceable cartridges.

12. The peritoneal dialysis system of claim 11, wherein the replaceable cartridges include: activated charcoal, urease, zirconium phosphate, hydrous zirconium oxide and activated carbon.

13. The peritoneal dialysis system of claim 1, wherein the sorbent devices are connected in parallel.

14. A method of continuous removal of impurities from the blood of a patient using a wearable peritoneal dialysis system, comprising the steps of:
infusing fresh dialysate into the peritoneal cavity of the patient so that the impurities in the blood are diffused across the peritoneal membrane and into the dialysate;
removing the spent dialysate from the peritoneal cavity; and
regenerating the dialysate using a plurality of sorbent devices that are worn on the body of a patient.

15. The method of claim 14, further comprising the step of connecting the sorbent devices in series.

16. The method of claim 14, further comprising the step of providing a flexible casing for each of the sorbent devices for conformity with the body contour of the patient.

17. The method of claim 14, further comprising the step of varying the number of sorbent devices to reflect different dialysis prescriptions.

18. The method of claim 14, further comprising the step of providing a regenerated dialysate inlet tube leading into the peritoneal dialyzer and a spent dialysate outlet tube leading out of the peritoneal dialyzer.

19. The method of claim 18, further comprising the step of providing a side port on the regenerated dialysate inlet tube for the infusion of additives.

20. The method of claim 19, further comprising the step of pumping the additives into the dialysate from a plurality of additive reservoirs.

21. The method of claim 18, further comprising the step of controlling the rate of infusion of each additive electronically.

22. The method of claim 18, further comprising the step of choosing the additives from the group consisting of: sodium citrate, calcium, potassium and sodium bicarbonate.

23. The method of claim 14, wherein the sorbent devices comprise replaceable cartridges.

24. The method of claim 23, wherein the replaceable cartridges include: activated charcoal, urease, zirconium phosphate, hydrous zirconium oxide and activated carbon.

25. The method of claim 14, further comprising the step of connecting the sorbent devices in parallel.
